(19) United States
(12) Patent Application Publication
(10) Pub. No.: US 2002/0187932 A1
(43) Pub. Date: Dec. 12, 2002

(54) Treatment of anorexia nervosa and bulimia

(76) Inventors: Roger Antonius Henricus Adan, Utrecht (NL); Tom Vink, Utrecht (NL)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/040,009

(22) Filed: Jan. 3, 2002

(30) Foreign Application Priority Data
May 18, 2001 (GB) 0112235.7

(51) Int. Cl. A61K 38/17

(52) U.S. Cl. 514/12

(57) ABSTRACT

According to the present invention, a method for the treatment of anoxeria nervosa (AN) or bulimia comprises administering to a patient having AN or bulimia an effective amount of an inverse agonist on MC4-r.

### FIELD OF THE INVENTION

This invention relates to the treatment of anorexia nervosa and bulimia.

### BACKGROUND OF THE INVENTION

Anorexia nervosa (AN) and bulimia are life-threatening disorders affecting mostly adolescent women. AN at least is a dramatic psychiatric syndrome accompanied by severe weight loss, hyperactivity and neuroendocrine changes. Several studies have shown a strong genetic component in AN; see, for example, Hebebrand and Remschmidt, Hum. Genet. 95:1-11 (1995).

Recent advances in unravelling the mechanisms of weight control point to a crucial role of the melanocortin-4 receptor (MC4-r) system in regulating body weight. See, for example, Salton et al., Neuron 25: 265-8 (2000).

The discovery of leptin as a starvation signal was one of the milestones in unravelling the signalling cascade regulating energy balance. Loss of function mutations in the leptin gene, as well as in the leptin receptor gene, result in obesity, both in humans and rodents. The central melanocortinergic system is one of the major pathways of leptin signalling, acting directly downstream of leptin. The central melanocortinergic system is constituted by at least two melanocortin receptors, melanocortin 3 receptor (MC3-r) and MC4-r, and by two classes of hormones. The melanocortins (like α-MSH and ACTH) are products of the pro-opiomelanocortin gene (POMC) and act as MC receptor agonists.

By contrast to melanocortins, agouti-related protein (AGRP) acts as an endogenous MC receptor inverse agonist. Leptin stimulates POMC expression and suppresses AGRP expression in the arcuate nucleus of the hypothalamus. Loss of function mutations of POMC or the MC4-r are associated with obesity in both mice and man while overexpression of AGRP, in transgenic mice, also results in obesity; see Salton et al., supra, and Shutter et al., Genes Dev. 11: 593-602 (1997).

In AN, plasma levels of leptin are low, signalling starvation to the brain. Low levels or absence of leptin upregulate AGRP expression in rodents; see Mizuno and Mobbs, Endocrinology 140: 814-7 (1999).

Rosse et al., Endocrinology 139: 4428-31 (1998), discloses that AGRP, when injected into the rodent brain, increases food intake and body weight. Thus, AGRP is one of the important orexigenic peptides regulating energy balance downstream of leptin.

In AN, decreased food intake is accompanied by hyperactivity and activation of the hypothalamo-pituitary adrenal (HPA) axis. Studies in rats have demonstrated that activation of MC4-r not only results in decreased food intake, but under specific conditions also in activation of the HPA axis and increased motor activity; see von Frijtag et al., Br. J. Pharmacol. 123: 1503-8 (1998).

Several candidate genes have been screened for association with anorexia nervosa, including the 5HT2A receptor gene, the 5HT transporter gene, the dopamine D3 and D4 receptor gene, the 5HT1D and 5HT7 receptor genes, the leptin gene, the MC4-r gene, the POMC gene, NPY-Y5 receptor gene, the estrogen receptor beta and the beta 3-adrenergic-receptor. Thus far, only a polymorphism in the 5HT2A receptor gene promoter has been shown to be associated with AN; however similar studies have failed to confirm this association. An association has been reported, by Campbell et al., Mol. Psychiatry 4: 68-70 (1999), between microsatellites flanking the UCP-2/UCP-3 gene cluster and AN.

### SUMMARY OF THE INVENTION

It has now been found that antagonism, and more specifically inverse antagonism, of MC4-r may be considered as pharmacotherapy for patients with AN. This discovery was made by determining the sequence of the coding region of the human AGRP gene (AGRP), and screening for variations in the AGRP of 100 patients with AN. Three single nucleotide polymorphisms (SNP's) were identified and screened in a further 45 patients and 244 controls. Two alleles were in complete linkage disequilibrium and were significantly enriched in anorectic patients (12.5%; p=0.0027) compared to controls (4.5%). These data indicate that variations of AGRP are associated with susceptibility for AN. Without wishing to be bound by theory, this is possibly caused by defective suppression of the MC4-r by the variant AGRP, leading to a decreased feeding signal, increasing the risk of developing AN. Indeed, detection of a gene polymorphism in the coding region of AGRP associated with AN, provides evidence that self-starvation results from insufficient suppression of central melanocortin activity. Furthermore, central infusion of AGRP ameliorates self-starvation, physical hyperactivity, deregulated body temperature, and therefore survival rate. These co-morbidities are all analogous to those associated with AN.

According to the present invention, a method for the treatment of AN or bulimia comprises administering to a patient having AN or bulimia an effective amount of an inverse agonist of MC4-r.

### DESCRIPTION OF PREFERRED EMBODIMENTS

Any inverse agonist of MC4-r may be used in this invention, provided that it has the desired activity. This activity may be determined by known assays. It is preferably at least 50% of the activity exhibited by AGRP.

Preferred agents for use in the invention have one or more characteristics of AGRP. Structurally, a fragment or variant of AGRP is preferably one having at least 50% identity to AgRP, and the agonist has at least 50% of the activity of AgRP with respect to MC4-r. A fragment consisting of amino acids 109-118 has been shown to have an efficacy similar to that of the whole protein.

A peptide of this invention may be cyclised, e.g. end-to-end or by substituting/introducing 2 Cys residues. Cyclisation procedures are known; see, for example, Tam et al, JACS 113:6657-62 (1991). Other cyclisations, e.g. Mitsunobu or olefin metathesis ring closure, may also be used. The cyclic peptides may exhibit enhanced properties.

Peptides of the invention include modifications of the given sequences. Such modifications are well known to those skilled in the art. Isosteric replacements include Abu for Cys (this may be desirable where the peptide should retain an even number of Cys residues for cyclisation), Phe for Tyr and different alkyl/aryl substituents. The shifting of substituents within an amino acid residue, from a C atom to a N atom, to produce peptoids having greater resistance to proteolysis, and other modifications, are known and are included within the scope of this invention.

By means of the invention, AN or bulimia in humans can be treated, e.g. controlled or prevented. For this purpose, the active compound can be formulated in any suitable manner, together with a conventional diluent or carrier, e.g. as a tablet, capsule, solution etc. The active compound may be administered by the oral, parenteral or other route. The effective amount of active agent to be administered can be readily determined by the skilled person, and will depend on the usual factor such as the frequency of dosing, the age and condition of the patient, the nature and degree of the complaint, the rate of administration, coadministered drugs etc. A daily dosage may be in the range of 0. 1-100 mg of the active agent.

The following experiments provide the evidence on which the invention is based.

Clones of B16/G4F cells, each expressing the MC4-r at a different level, were tested for basal (unstimulated) and forskolin-induced AC (adenylate cyclase) activity. The response of a receptor, and this correlation was observed with the expression levels of the MC4-r. The clone with the highest expression of the MC4-r also showed higher basal AC activity. These data clearly suggest that there is constitutive activity of the MC4-r.

AGRP reduced (dose-dependently) both basal and forskolin-induced AC activity only in cells expressing the MC4-r. The effect was most profound in cells with high expression levels (and constitutive activity). Thus, AGRP suppresses constitutive activity.

Human AGRP was amplified from gDNA and fully sequenced (Genbank: AF28 1309). The gene contains three small introns comparable to the mouse gene and having the structure as described for the human gene, as reported by Shutter et al., supra. Furthermore, the sequence was virtually identical to a draft sequence of a human chromosome 16 clone (Genbank: AC009061). gDNAfrom 100 patients with AN was amplified and sequenced, identifying three polymorphisms. Two polymorphisms were located in exons. One of the polymorphisms, G760A (introducing a Hinfl restriction site) in exon 3, leads to the amino acid substitution Ala67Thr in AGRP. The exon 2 polymorphism, G526A, is silent and is not part of a splice site (intron 2 starts at position 534). The third polymorphism in intron 2, C650T, does not appear to involve any splice or gene regulatory sequences.

A further 84 patients and 244 controls were screened for the presence of these polymorphisms, using either Hinfl restriction digestion of PCR fragments for the 760A allele and ASO probing for the 526A and 650T alleles. All subjects with 760A also had 526A, and these two polymorphisms seem to be in complete linkage disequilibrium. None of the subjects with these two polymorphisms had the third 650T polymorphism.

The frequencies of the polymorphisms are indicated in Table 1. The frequency of the 760A1526A haplotype was increased in patients with AN as compared to controls (p=0.0027; Table 1). When corrected for multiple testing, the increase in allele frequency of the 760A1 526A haplotype was still significant (p=0.0054). The 760A/526A haplotype was distributed equally amongst gender and restricting versus purging type. The frequency of the 650T haplotype was similar in AN patients and controls.

More specifically, 184 patients with AN fulfilling the DSM-IV criteria (see American Psychiatric Association (1994) “Diagnostic and statistical manual of mental disorders”, 4th ed., Washington, USA) were recruited at the Department of Child and Adolescent Psychiatry of the University of Marburg, Germany, and the Department of Child and Adolescent Psychiatry, University Medical Center, Utrecht, The Netherlands, respectively. According to DSM-IV criteria, from 184 patients with AN, 113 were of the restricting type and 32 of the purging type. 100 volunteers functioned as the German controls. 144 volunteers functioned as the Dutch controls. Written informed consent was given by all participants or, in case of minors, their parents. The investigation was approved by the ethics committees of the University of Marburg and the University Medical Center Utrecht.

The coding region of the human AGRP was amplified from gDNA using primers based on the cDNA sequence and directly sequenced using a CEQ 2000 capillary sequencer (Beckman) and a dye terminator sequencing kit (Beckman). For haplotype analysis on additional samples, another PCR was done amplifying a 533 bp part (348-880, containing all SNP's). Haplotyping was done either by Hinfl restriction digestion (G760A) or by allele specific oligonucleotide hybridisation (G526A and C650T). For the three detected polymorphisms, the genotype frequencies in patients and controls were compared via Fisher's exact test, against the one-sided alternative that the mutant allele was more frequent in AN patients. The procedure followed in the molecular analysis (to screen for mutations in patients only, and to subsequently test the controls for all polymorphisms that were detected in the patient population) introduces a bias. This bias can be quantified as follows: of all possible 2×2 tables with the observed marginal totals, the table in which the mutant allele would have a frequency of 0 in patients will never reach the analysis stage. If pO represents the hypergeometric probability of that table, then a correction for the (small) bias can be obtained by multiplying the calculated P-value by 1/(1-p0). Significance calculations were also carried out via a Mantel-Haenszel chi-square test, conditional on nationality (German or Dutch). Results were virtually identical to the results of the combined analysis (data not shown). Bonferroni's correction for multiple testing was applied. Analyses for the two polymorphisms that appeared in complete linkage disequilibrium were not considered as independent tests. Genotype frequencies in controls were in almost complete agreement with Hardy-Weinberg expectations for all variants.

TABLE 1
Heterozygote frequencies of the AGRP polymorphisms

| | Heterozygote Frequencies | |
|---|---|---|
| | 760 G/A | |
| | 526 G/A | 650 C/T |
| Patients with Anorexia Nervosa | 23/184 (12.5%) | 5/184 (3/4%) |
| Controls | 11/244 (4/5%) | 9/244 (3.7%) |
| p value | 0.0027 | 0.89 |

Statistical analysis was done by comparing the genotype frequencies between the patient group and the control group. Frequencies refer to numbers of carriers and total numbers of tested subjects. Significance calculations were based on allele counts. Homozygotes for the SNP's were not detected.

The new results, after screening only the coding area of AGRP, put forward allelic variation of the AGRP as a risk factor for AN. These results indicate that the 760A mutation conveys a relative risk (as approximated via calculation of an odds ratio) of 3.0 for carriers to develop AN. Screening of the AGRP gene exons by SSCP analysis in 290 obese patients was described by Vaisse et al., J. Clin. Invest. 106: 253-62 (2000). Interestingly, the 520A polymorphism (named G423A) was detected, but only in one patient, indicating a frequency of 0.3% in the obese patient sample. The 760A SNP was not detected in this patient, but that SNP is on a different exon and could have escaped the SSCP analysis. If this observation is confirmed, it would indicate that the 760A SNP is negatively associated with obesity. Al a67 is in the middle of the AGRP protein, in front of the C-terminal Cys-rich part of the protein with receptor binding activity. This part of the molecule has not previously been implicated in the receptor-binding activity of AGRP, which presumably resides in the Cys-rich C-terminal part of the protein. Nevertheless, the change of Ala to Thr is a non-conservative substitution and could result in destabilisation and/or dysfunction of the protein. Interestingly, Ala67 is located only 2 amino acids from the proteolytic site at position 69, which generates a fragment of AGRP with higher affinity than the full-length protein.

AGRP mRNA levels in rodents are increased when food is restricted. AGRP is an orexigenic peptide inhibiting the activity of the melanocortinergic system. Inadequate blockade of the melanocortinergic system during fasting, due to a mutation of AGRP, would result in lower stimulation of food intake. This might explain that mutations in AGRP increase the susceptibility to develop AN specifically during periods of food restriction, when AGRP mRNA levels are normally increased in the hypothalamus. This finding points to a role of the leptin/melanocortinergic signalling pathway in the pathophysiology of AN.

Central infusion of AGRP, a suppressor of melanocortin activity, ameliorates self-starvation, physical hyperactivity, and deregulated body temperature, and therefore survival rate. These co-morbidities are all analogous to those associated with AN in humans. Furthermore, there is an association between an AGRP gene polymorphism and AN. This gene polymorphism in the coding region of the AGRP gene changes the structure of the AGRP protein. Therefore, it is proposed that insufficient suppression of increased receptor activity (by AGRP) contributes to emaciation. Therefore, antagonism, and more specifically inverse antagonism, of MC4-r may be considered as pharmacotherapy for patients with AN and also bulimia.

What is claimed is:

1. A method for the treatment of a subject exhibiting anorexia nervosa or bulimia, which comprises administering to the subject an inverse agonist of the melanocortin 4 receptor (MC4-r).

2. The method according to claim 1, wherein the inverse agonist is Agouti-Related Protein (AGRP) or a fragment thereof, or a variant thereof having at least 50% identity to AGRP, and the agonist has at least 50% of the activity of AGRP with respect to MC4-r.

3. The method according to claim 1, wherein the inverse agonist is AGRP.
